(19) United States
(12) Patent Application Publication
(10) Pub. No.: US 2017/0129893 A1
(43) Pub. Date: May 11, 2017

(54) Novel Crystalline Form of Levomefolate

(71) Applicant: Mylan Laboratories Limited, Hyderabad (IN)

(72) Inventors: Madhuresh Kumar Sethi, Hyderabad (IN); Vijendra Singh Rawat, Hyderaba (IN); Jaganmohana Rao Bontalakoti, Hyderaba (IN); LKakshminarayana Vemula, Hyderaba (IN)

(21) Appl. No.: 15/319,223

(22) PCT Filed: Jun. 12, 2015
(86) PCT No.: PCT/IB2015/054456

§ 371 Date: Dec. 15, 2016

(30) Foreign Application Priority Data
Jun. 16, 2014 (IN) 2933CHE2014

(51) Int. Cl. C07D 475/04

(52) U.S. Cl. CPC C07D 475/04; C07B 2200/13

(57) ABSTRACT

The present disclosure relates to crystalline forms of levomefolate calcium). The present disclosure also relates to a process for the preparation of crystalline forms of levomefolate calcium.

### COGNATE APPLICATION

The specification below is a cognate application of Indian Patent Application no. 2933/CHE/2014 dated Jun. 16, 2014 and Indian Patent Application no. 3762/CHE/2014 dated Jul. 31, 2014.

### CROSS-REFERENCE TO RELATED APPLICATIONS

This application claims the benefit of the earlier filing date of Indian Provisional Patent Application No. 2933/CHE/2014 filed on Jun. 16, 2014; Indian Provisional Patent Application No. 3762/CHE/2014 filed on Jul. 31, 2014.

### BACKGROUND OF THE INVENTION

Field of the Invention

The present disclosure relates generally to novel crystalline forms of levomefolate calcium. The present disclosure also relates to processes for the preparation of crystalline forms of levomefolate calcium.

Background of the Invention

Levomefolic acid is the primary biologically active form of folic acid. The calcium salt of levomefolic acid, levomefolate calcium, is used in the treatment of megaloblastic folic acid deficiency anemia. Levomefolate calcium may also be used in conjunction with folate antagonists in cancer chemotherapy, such as aminopterin and methotrexate, to counteract the ill-effects of the folate antagonists. Levomefolate calcium is also incorporated into birth control formulations, such as BEYAZ® and SAFYRAL® (Bayer), to prevent a rare birth defect that could occur in a baby if a pregnancy occurs while taking birth control or shortly after stopping.

Levomefolate calcium is chemically known as N-[4-[[(2-amino-1,4,5,6,7,8-hexahydro-5-methyl-4-oxo-(6S)-pteridinyl)methyl]amino]benzoyl]-L-glutamic acid calcium salt and has the following structure:

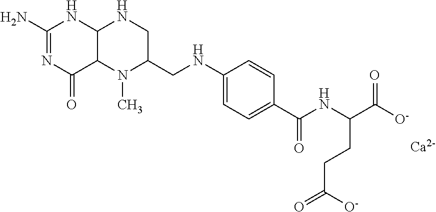

Levomefolate and pharmaceutically acceptable salts are disclosed in G.B. Patent No. 1,572,137, which is hereby incorporated by reference. U.S. Pat. No. 5,124,452 and U.S. Pat. No. 5,223,500, which are both also hereby incorporated by reference, disclose crystalline pentahydrate and amorphous forms of levomefolate calcium.

U.S. Pat. No. 6,441,168, which is hereby incorporated by reference, discloses four crystalline modifications of levomefolate calcium namely Type-I, Type-II, Type-III and Type-IV.

PCT Publication No. WO2013107236A1 (which is hereby incorporated by reference) discloses crystalline levomefolate calcium form-C and a process for the preparation of crystalline levomefolate calcium form-C, wherein levomefolate calcium is crystallized from a water medium in an ultrasonic reactor.

The present invention provides novel crystalline forms of levomefolate calcium and processes for the preparation thereof.

### SUMMARY OF THE INVENTION

One aspect of the present invention provides novel crystalline levomefolate calcium form-M.

Within the context of the present invention, the crystalline levomefolate calcium form-M may be characterized by the powdered X-ray diffraction (PXRD) pattern shown in FIG. 1.

Another aspect of the present invention provides a process for the preparation of crystalline levomefolate calcium form-M which may include the following steps:

a) dissolving levomefolate calcium in water to create a solution;

b) adding an anti-solvent to the solution; and

c) isolating crystalline levomefolate calcium form-M.

Yet another aspect of the present invention provides a process for the preparation of crystalline levomefolate calcium form-M which may include the following steps:

a) dissolving amorphous levomefolate calcium in water to create a solution;

b) adding an anti-solvent to the solution at 30-60° C.;

c) heating the solution to reflux at 60-70° C.;

d) cooling the solution to 20-30° C.; and

e) isolating crystalline levomefolate calcium form-M.

### BRIEF DESCRIPTION OF THE DRAWINGS

Further aspects of the present disclosure together with additional features contributing thereto and advantages accruing therefrom will be apparent from the following description of preferred embodiments of the disclosure shown in the accompanying drawing figures wherein:

FIG. 1 is a PXRD pattern of crystalline levomefolate calcium form-M.

FIG. 2 is a PXRD pattern of crystalline levomefolate calcium form-M1.

FIG. 3 is a PXRD pattern of crystalline levomefolate calcium form-M2.

FIG. 4 is a PXRD pattern of crystalline levomefolate calcium form-M3.

FIG. 5 is a PXRD pattern of crystalline levomefolate calcium form-M4.

### DETAILED DESCRIPTION OF THE DISCLOSURE

It is to be understood that the description of the present invention has been simplified to illustrate elements that are relevant for a clear understanding of the invention, while eliminating, for purposes of clarity, other elements that may be well known.

The present disclosure provides crystalline levomefolate calcium forms-M, M1, M2, M3, and M4. The present disclosure also relates to processes for the preparation of crystalline levomefolate calcium forms-M, M1, M2, M3, and M4.

One aspect the present invention provides crystalline levomefolate calcium form-M.

One embodiment of the present disclosure provides a process for the preparation of crystalline levomefolate calcium form-M which may include the following steps:

a) dissolving levomefolate calcium in water to make a solution;

b) adding an anti-solvent to the solution; and

c) isolating crystalline levomefolate calcium form-M.

According to the present invention, levomefolate calcium may be dissolved in water. An anti-solvent may then be added to the solution at the same temperature. The solution may then be heated to reflux for about 1 to 2 hours and then cooled. The obtained solid may then be isolated, for example, by filtering, to yield crystalline levomefolate calcium form-M.

Within the context of the present invention, the anti-solvent may be a liquid ketone solvent. Examples of suitable ketone solvents include acetone, methyl ethyl ketone, diisopropyl ketone, methyl tert-butyl ketone, and mixtures thereof. In some embodiments, the anti-solvent is acetone. One of skill in the art will recognize other ketone solvents that may be employed.

Another embodiment of the present invention provides a process for the preparation of crystalline levomefolate calcium form-M which may include the following steps:

a) dissolving an amorphous levomefolate calcium in water to create a solution;

b) adding an anti-solvent to the solution at 30-60° C.;

c) heating the solution to reflux at 60-70° C.;

d) cooling the solution to 20-30° C.; and

e) isolating crystalline levomefolate calcium form-M.

According to the present disclosure, amorphous levomefolate calcium may be dissolved in water at a temperature of about 40° C. to 50° C. An anti-solvent may then be added to the solution at the same temperature, heated to reflux at about 60° C. to 70° C. for about 1 to 2 hours and then cooled to 20° C. to 30° C. The obtained solid may then be isolated, for example, by filtering, to yield crystalline levomefolate calcium form-M.

Within the context of the present disclosure, the anti-solvent may be a ketones. Examples of suitable ketone solvents include acetone, methyl ethyl ketone, diisopropyl ketone, methyl tert-butyl ketone, and mixtures thereof. In one embodiment, the anti-solvent is acetone. One of skill in the art will recognize other ketone solvents that may be employed.

The polymorphs of the present disclosure may be characterized by their powder X-ray diffraction (PXRD) patterns. Thus, the X-ray diffraction patterns of said polymorphs of the disclosure were measured.

The PXRD patterns were measured on a BRUKER D-8 Discover powder diffractometer equipped with goniometer of 0/20 configuration and Lynx Eye detector. The Cu-anode X-ray tube was operated at 40 kV and 30 mA. The experiments were conducted over the 20 range of 2.0°-50.0°, 0.030° step size and 0.4 seconds step time.

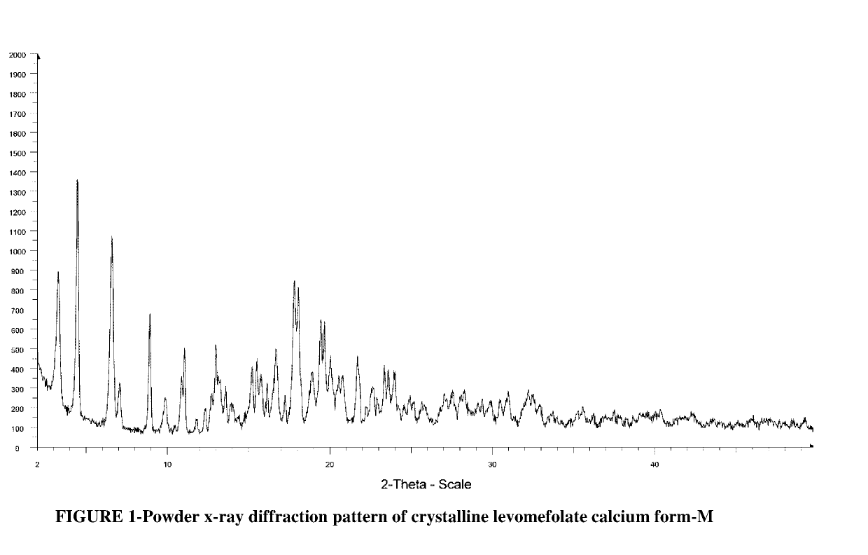

According to the present invention, crystalline levomefolate calcium form-M may be characterized by the PXRD pattern shown in FIG. 1.

Within the context of the present invention, the crystalline levomefolate calcium form-M polymorph may be further characterized by a PXRD pattern having significant peaks at 2θ angle positions at about 3.34, 4.53, 6.64, 7.10, 17.88 and 18.13±0.2°.

Within the context of the present invention, crystalline levomefolate calcium form-M may be further characterized a PXRD patterns having significant peaks at 2θ angle positions of about 3.34, 4.53, 6.64, 7.10, 8.98, 9.93, 10.98, 11.12, 11.88, 12.39, 12.79, 13.74, 13.30, 13.66, 14.03, 14.40, 15.26, 15.58, 15.84, 16.22, 16.80, 17.32, 17.88, 18.13, 18.99, 19.53, 20.14, 20.89, 21.81, 22.35, 22.76, 23.72, 24.09, 25.04, 25.88, 27.25, 27.63, 28.15, 28.40, 30.00, 30.62, 31.08, 32.31, 32.58 and 32.99±0.2°.

Another aspect of the present invention provides novel crystalline levomefolate calcium form-M₁.

One embodiment of the present invention provides a process for the preparation of crystalline levomefolate calcium form-M1, which can be achieved by heating crystalline levomefolate calcium form-M at about 80° C. to about 100° C. under nitrogen atmosphere for about 10 to 30 minutes.

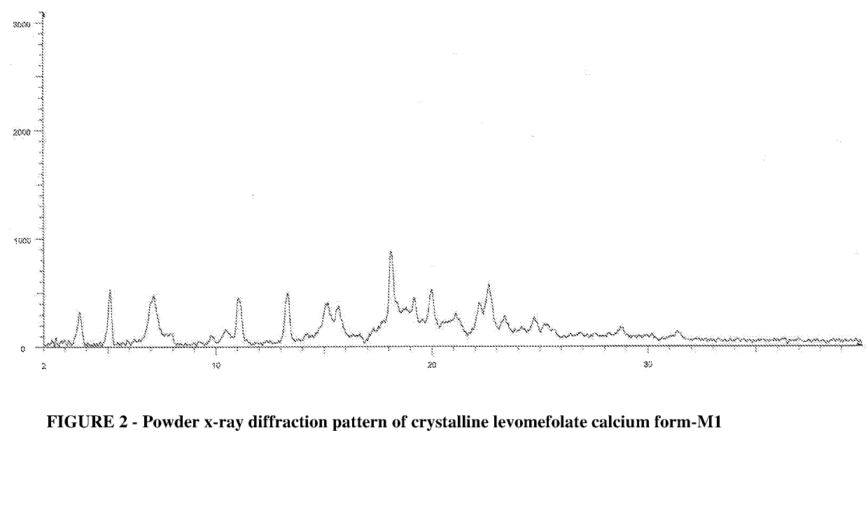

According to the present invention, crystalline levomefolate calcium form-M1 may be characterized by the PXRD pattern shown in FIG. 2.

Within the context of the present invention, crystalline levomefolate calcium form-M1 may be further characterized by a PXRD pattern having significant peaks at 20 angle positions at about 3.64, 5.05, 7.09, 9.78, 10.44, 11.04, 13.29, 15.13, 15.64, 18.11, 19.17, 19.98, 21.11, 22.22, 22.64, 23.37, 24.74, 28.79 and 31.38±0.2°.

Another aspect of the invention provides crystalline levomefolate calcium form-M2.

One embodiment of the present invention provides a process for the preparation of crystalline levomefolate calcium form-M2, which may be achieved by heating the crystalline levomefolate calcium form-M at about 80° C. to about 100° C. for about 10 to 30 minutes.

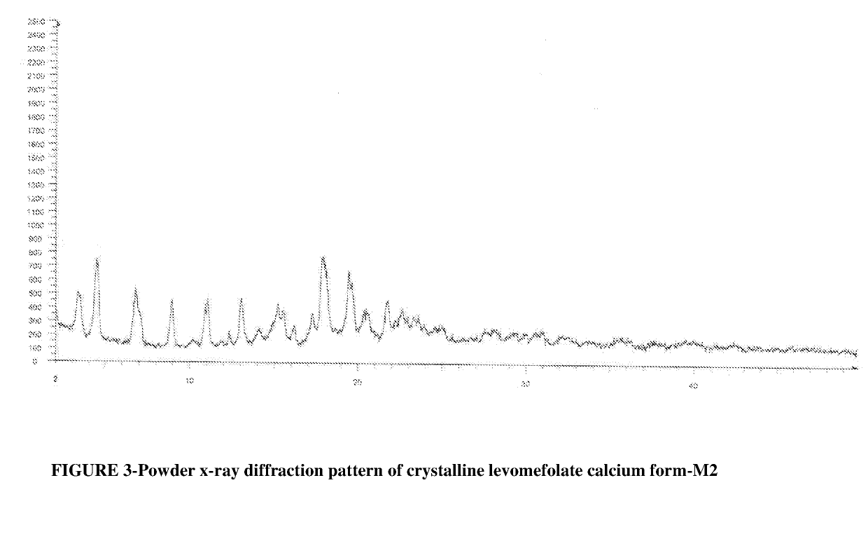

According to the present invention, crystalline levomefolate calcium form-M2 may be characterized by the PXRD pattern shown in FIG. 3.

Within the context of the present invention, crystalline levomefolate calcium form-M2, may be further characterized by a PXRD pattern having significant peaks at 2θ angle positions at about 3.33, 4.43, 5.81, 6.75, 7.03, 8.90, 10.19, 11.01, 11.84, 12.30, 13.00, 14.05, 15.18, 15.47, 16.14, 17.26, 17.87, 19.46, 20.39, 21.73, 22.62, 24.99, 27.54, and 30.98±0.2°.

Another aspect of the present invention provides a crystalline levomefolate calcium form-M3.

One embodiment of the present invention provides a process for the preparation of crystalline levomefolate calcium form-M3, which may be achieved by heating the crystalline levomefolate calcium type-I at about 80° C. to 100° C. under nitrogen atmosphere for about 10 to 30 minutes.

Within the context of the present disclosure, levomefolate calcium type-I may be prepared according the process disclosed in U.S Pat. No. 6,441,168.

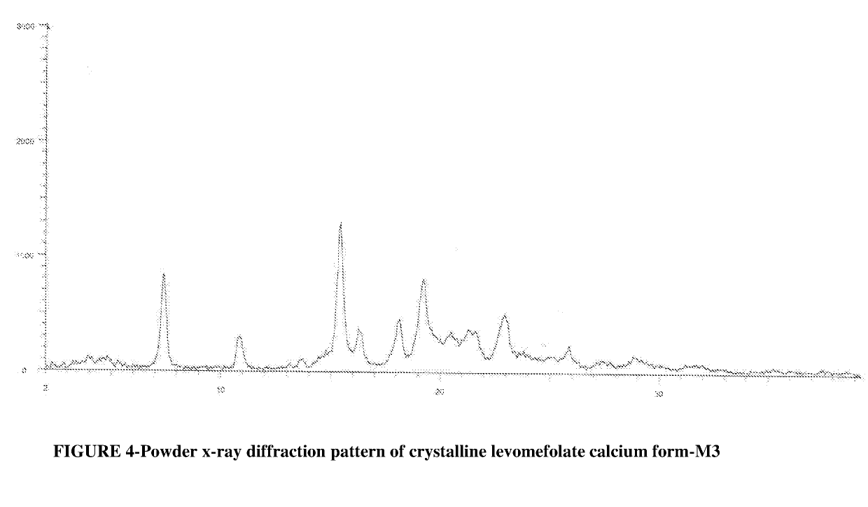

According to the present invention, crystalline levomefolate calcium form-M3 may be characterized by the PXRD pattern shown in FIG. 4.

Within the context of the present invention, crystalline levomefolate calcium form-M3 may be further characterized by a PXRD pattern having significant peaks at 2θ angle positions at about 3.95, 7.36, 10.82, 13.11, 13.67, 15.41, 16.29, 18.11, 19.23, 20.48, 21.33, 21.60, 22.93, and 25.87±0.2°.

Another aspect of the present invention provides crystalline levomefolate calcium form-M4.

One embodiment of the present invention provides a process for the preparation of crystalline levomefolate calcium form-M4, which may be achieved by heating the crystalline levomefolate calcium form-C at about 80° C. to 100° C. under nitrogen atmosphere for about 10 to 30 minutes.

Within the context of the present disclosure, levomefolate calcium form-C may be prepared according to the process disclosed in PCT Publication No. WO2013107236A1.

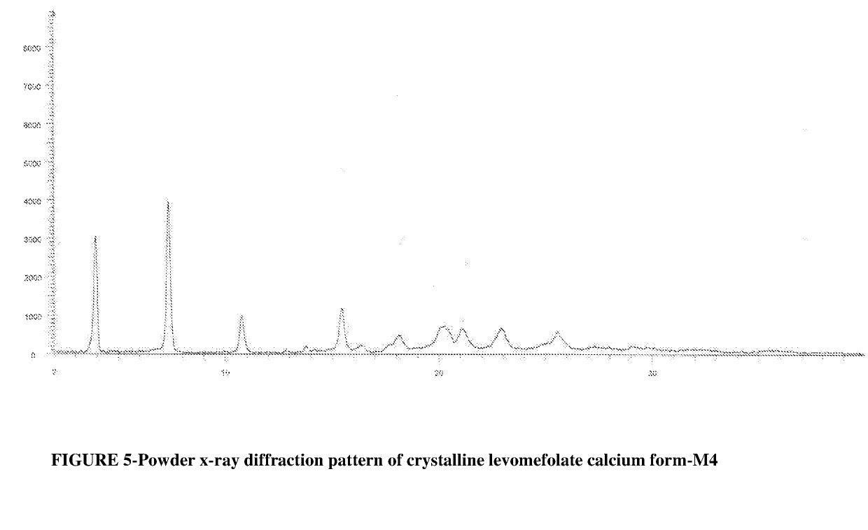

According to the present invention, crystalline levomefolate calcium form-M₄ may be characterized by the PXRD pattern shown in FIG. 5.

Within the context of the present invention, crystalline levomefolate calcium form-M4 may be further characterized by a PXRD pattern having significant peaks at 2θ angle positions at about 3.89, 7.32, 10.74, 12.82, 13.73, 14.12, 15.41, 16.35, 17.67, 18.13, 20.15, 21.08, 22.93, 25.57, and 29.05±0.2°.

The levomefolate calcium polymorphs disclosed herein may be included in formulations prescribed for the treatment of megaloblastic folic acid deficiency anemia, in formulations to be prescribed in conjunction with folate antagonists, for example, aminopterin and methotrexate, or be included in formulations of birth control. Formulations containing levomefolate calcium may include a variety of excipients that would be known to one skilled in the art to achieve a final dosage form. The levomefolate calcium polymorphs disclosed herein may be formulated as a solid dosage form, such as a capsule or a tablet, for administration to patients.

Certain specific aspects and embodiments of the present application will be explained in greater detail with reference to the following examples. The examples are provided only for purposes of illustration and should not be construed as limiting the scope of the disclosure in any manner. Reasonable variations of the described procedures are intended to be within the scope of the present application. While particular aspects of the present application have been illustrated and described, it would be apparent to those skilled in the art that various other changes and modifications can be made without departing from the spirit and scope of the disclosure. It is therefore intended to encompass all such changes and modifications that are within the scope of this disclosure.

### EXAMPLE 1

### Preparation of Crystalline Levomefolate Calcium Form-M

Amorphous levomefolate calcium (5 g) was suspended in water (50 mL) at room temperature under nitrogen atmosphere. The solution was heated to 40-50° C. to achieve a clear solution. Acetone (50 mL) was added slowly to the clear solution at 40-50° C. The solution was then heated to reflux at 60-70° C. for one hour and then cooled to 20-30° C. The crystalline material was recovered from filtration followed by washing with acetone (10 mL) and further dried under vacuum at 35-40° C. to get levomefolate calcium form-M (3.2 g).

### EXAMPLE 2

### Preparation of Crystalline Levomefolate Calcium Form-M

N-[4-({[(6S)-2-amino-5-methyl-4-oxo-3,4,5,6,7,8-hexahydropteridin-6-yl]methyl}amino) benzoyl]-L-glutamic acid-1-[(2S)-1-ethylpyrrolidin-2-yl]methanamine (10 g) was suspended in water (60 mL) and calcium chloride dihydrate (5 g) was added at room temperature under nitrogen atmosphere. The solution was heated to 35-40° C. to get a clear solution and then cooled to 15±5° C. The material was recovered by filtration followed by washing with water (30 mL) and further dried under vacuum at 40-45° C. The dry material was suspended in water (50 mL) at 20-25° C. under nitrogen atmosphere. The solution was heated to 40-50° C. to get a clear solution. Acetone (50 mL) was added slowly to the clear solution at 40-50° C. The resulting solution was heated to reflux at 60-70° C. for 2 hours and then cooled to 20-30° C. The material was recovered by filtration followed by washing with acetone (10 mL) and further dried under vacuum at 35-40° C. to get crystalline levomefolate calcium form-M (5 g).

### EXAMPLE 3

### Preparation of Crystalline Levomefolate Calcium Form-M

Crystalline levomefolate calcium (5 g) was suspended in water (50 mL) at room temperature under nitrogen atmosphere. The solution was heated to 40-50° C. to achieve a clear solution. Acetone (50 mL) was added slowly to the clear solution at 40-50° C., the solution was then heated to reflux at 60-70° C. for one hour and then cooled to 20-30° C. The crystalline material was recovered from filtration followed by washing with acetone (10 mL) and further dried under vacuum at 35-40° C. to get crystalline levomefolate calcium form-M (3.2 g).

### EXAMPLE 4

### Preparation of Crystalline Levomefolate Calcium Form-M1

Levomefolate calcium form-M (4 g) was heated to 80-100° C. under nitrogen atmosphere for 10-30 minutes to get levomefolate calcium form-M1 (3 g).

### EXAMPLE 5

### Preparation of Crystalline Levomefolate Calcium Form-M2

Levomefolate calcium form-M (4 g) was heated to 80-100° C. for 10-30 minutes to get levomefolate calcium form-M2 (2.3 g).

### EXAMPLE 6

### Preparation of Crystalline Levomefolate Calcium Form-M3

Levomefolate calcium type-I (4 g) was heated to 80-100° C. under nitrogen atmosphere for 10-30 minutes to get crystalline levomefolate calcium form-M3 (2.5 g).

### EXAMPLE 7

### Preparation of Crystalline Levomefolate Calcium Form-M3

Levomefolate calcium type-I (4 g) was heated to 80-100° C. for 10-30 minutes to get crystalline levomefolate calcium form-M3 (2. g).

### EXAMPLE 8

### Preparation of Crystalline Levomefolate Calcium Form-M4

Levomefolate calcium crystalline form-C (4 g) was heated to 80-100° C. under nitrogen atmosphere for 10-30 minutes to get levomefolate calcium form-M4 (2.8 g).

### EXAMPLE 9

### Preparation of Crystalline Levomefolate Calcium Form-M4

Levomefolate calcium crystalline form-C (4 g) was heated to 80-100° C. for 10-30 minutes to get levomefolate calcium form-M4 (3.0 g).

What is claimed is:

1) Crystalline levomefolate calcium form-M.

2) The crystalline levomefolate calcium form-M of claim 1, which has a powder X-ray diffraction pattern having significant peaks at 2θ angle positions at about 3.34, 4.53, 6.64, 7.10, 17.88 and 18.13±0.2°.

3) The crystalline levomefolate calcium form-M of claim 1, which has a powder X-ray diffraction pattern as shown in FIG. 1.

4) A process for the preparation of crystalline levomefolate calcium form-M comprising the steps of:
a) dissolving levomefolate calcium in water to create a solution;
b) adding anti-solvent to the solution; and
c) isolating crystalline levomefolate calcium form-M.

5) The process according to claim 4, wherein the anti-solvent is a ketone.

6) The process according to claim 5, wherein the ketone is selected from the group acetone, methyl ethyl ketone, diisopropyl ketone, methyl tert-butyl ketone, and mixtures thereof.

7) A process for the preparation of crystalline levomefolate calcium form-M comprising the steps of:
a) dissolving an amorphous levomefolate calcium in water to form a solution;
b) adding an anti-solvent to the solution at 30-60° C.;
c) heating the solution to reflux at 60-70° C.;
d) cooling the solution to 20-30° C.; and
e) isolating crystalline levomefolate calcium form-M.

8) The process according to claim 7, wherein the anti-solvent employed is a ketone.

9) The process according to claim 8, wherein the ketone is selected from the group consisting of acetone, methyl ethyl ketone, diisopropyl ketone, methyl tert-butyl ketone, and mixtures thereof.
